(19) United States
(12) Patent Application Publication
(10) Pub. No.: US 2025/0195325 A1
(43) Pub. Date: Jun. 19, 2025

(54) MASSAGERS AND METHODS OF USING THE LIKE

(71) Applicant: JeanAne Hecht, Washington, DC (US)

(72) Inventor: JeanAne Hecht, Washington, DC (US)

(21) Appl. No.: 18/980,434

(22) Filed: Dec. 13, 2024

Related U.S. Application Data
(60) Provisional application No. 63/610,741, filed on Dec. 15, 2023.

(51) Int. Cl. A61H 23/02; A61N 5/00

(52) U.S. Cl. CPC A61H 23/0254; A61H 2201/0153; A61H 2201/1215; A61N 5/00

(57) ABSTRACT

A massager comprising a base having a first surface opposite a second surface, a first side opposite a second side, and a concave wing extending in a direction opposite the first surface and defining the first side. An elongate massage member connecting to the second surface of the base and extending in a direction away from the second surface and extending from the first side to the second side. A motor disposed in the base and adapted to produce vibration that is transferred through the base to the elongate massage member. An extension having a first end extending from the first surface of the base in a direction opposite the second surface of the base and a grip positioned at a second end of the extension. A switch disposed in the grip and having an on and an off position configured to turn the motor on and off.

### FIELD

The disclosure relates to the field of personal devices. More particularly, the disclosure relates to personal massagers useful in the massaging of tissue, such as tissue subjected to lymphadenectomy, radiation treatment, or both. Specific examples described in the disclosure relate to massagers useful in self treatment of lymphedema, enhancing lymphatic drainage, or both and methods of performing self massage, such as methods of performing self treatment of lymphedema, enhancing lymphatic drainage, or both.

### BACKGROUND

Lymphedema is a chronic condition characterized by the accumulation of lymphatic fluid in the interstitial spaces, resulting in swelling, discomfort, and impaired limb function. This condition often occurs as a consequence of surgical procedures, such as lymphadenectomy, commonly referred to as lymph node dissection, and radiation therapy. Existing therapeutic options for lymphedema management include manual lymphatic drainage, compression therapy, and exercise. However, these conventional options often fail to provide relief to individuals suffering from lymphedema. there remains a need for an innovative and effective solution that combines various therapeutic modalities to enhance patient outcomes.

A need exists, therefore, for improved devices and methods for the treatment of lymphedema, enhancing lymphatic drainage, or both.

### BRIEF SUMMARY OF SELECTED EXAMPLES

Various example massagers are described.

An example massager comprises a base having a first surface, a second surface, a first side and a second side; the first surface opposite the second surface and the first side opposite the second side; the base further having a concave wing defining the first side and extending in a direction opposite the first surface; an elongate massage member connecting to the second surface of the base and extending from the first side of the base to the second side of the base in a direction away from the second surface; a motor disposed in the base, the motor adapted to produce vibration that is transferred through the base to the elongate massage member; an extension having a first end and a second end, the first end of the extension extending from the first surface of the base in a direction opposite the second surface of the base; a grip positioned at the second end of the extension; and a switch disposed in the grip, the switch having an on and off position and configured to energize the motor when in the on position and to deenergize the motor when in the off position.

Another example massager comprises a base. The base having a first surface, a second surface, a first side, and a second side. The first surface opposite the second surface. The first side opposite the second side. The base having a first concave wing and a second concave wing. The first concave wing defining the first side and the second concave wing defining the second side. The first concave wing and the second concave wing extending in a direction opposite the first surface. A plurality of elongate massage members connecting to the second surface of the base, the plurality of elongate massage members extending in a direction away from the second surface and extending away from the first side of the base to the second side of the base. A motor disposed in the base. The motor adapted to produce vibration that is transferred through the base to the plurality of elongate massage members. An extension having a first end and a second end, the first end of the extension extending from the first surface of the base in a direction opposite the second surface of the base. A grip positioned at the second end of the extension. A switch disposed in the grip. The switch having an on and off position. The switch configured to turn the motor on when in the on position and to turn the motor off when in the off position.

An example performing self treatment of lymphedema or a radiated area comprises placing a massager at the affected area, the massager comprising a base. The base having a first surface, a second surface, a first side, and a second side. The first surface is opposite the second surface. The first side is opposite the second side. The base having a first concave wing and a second concave wing. The first concave win defining the first side, and the second concave wing defining the second side. The first concave wing and the second concave wing extending in a direction opposite the first surface. A plurality of elongate massage members connecting to the second surface of the base. The plurality of elongate massage members extending in a direction away from the second surface and extending from the first side of the base to the second side of the base. A motor disposed in the base. The motor adapted to produce vibration that is transferred through the base to the plurality of elongate massage members. An extension having a first end and a second end. The first end of the extension extending from the first surface of the base in a direction opposite the second surface of the base. A grip positioned at the second end of the extension. A switch disposed in the grip. The switch having an on and off position. The switch configured to turn the motor on when in the on position and to turn the motor off when in the off position. The method can further include: choosing a speed of vibration; turning the massager on; applying pressure to the massager; and massaging the affected area with the massager.

Additional understanding of the inventive massagers and methods can be obtained by reviewing the detailed description of selected examples, below, and the referenced drawings.

### DESCRIPTION OF FIGURES

FIG. 1 is a side view of a first example massager.

FIG. 2 is a top view of the first example massager.

FIG. 3 is a bottom view of the first example massager.

FIG. 4 is a side view of a second example massager.

FIG. 5 is a top view of the second example massager.

FIG. 6 is a bottom view of the second example massager.

FIG. 7 is a side view of a third example massager.

FIG. 8 is a top view of the third example massager.

FIG. 9 is a bottom view of the third example massager.

FIG. 10 is a side view of a fourth example massager.

FIG. 11 is a top view of the fourth example massager.

FIG. 12 is a bottom view of the fourth example massager.

FIG. 13 is a side view of a fifth example massager.

FIG. 14 is a bottom view of the fifth example massager.

FIG. 15 is a side view of the fifth example massager.

FIG. 16 is a flow chart of a performing self treatment of lymphedema or radiated area.

FIG. 17 is a side view of a sixth example massager.

FIG. 18 is a top view of the sixth example massager.

FIG. 19 is a bottom view of the sixth example massager.

FIG. 20 is a perspective view of the sixth example massager.

### DETAILED DESCRIPTION OF SELECTED EXAMPLES

The following detailed description and the appended drawings describe and illustrate various example massagers and methods of using the like. The description and illustration of these examples enable one skilled in the art to make and use examples of massagers. They do not limit the scope of the claims in any manner.

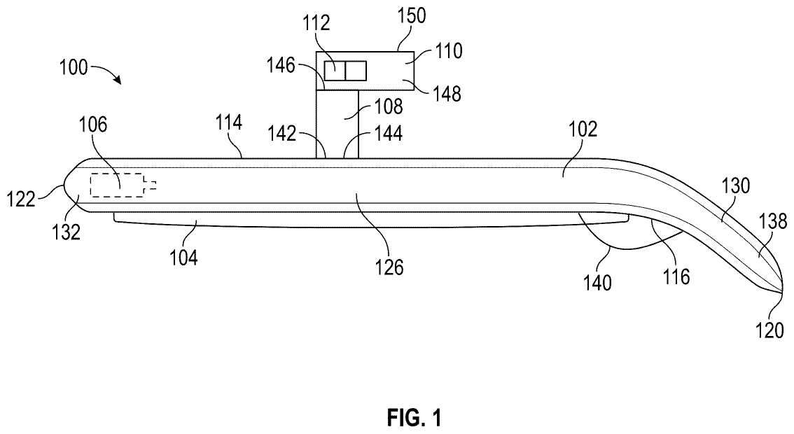

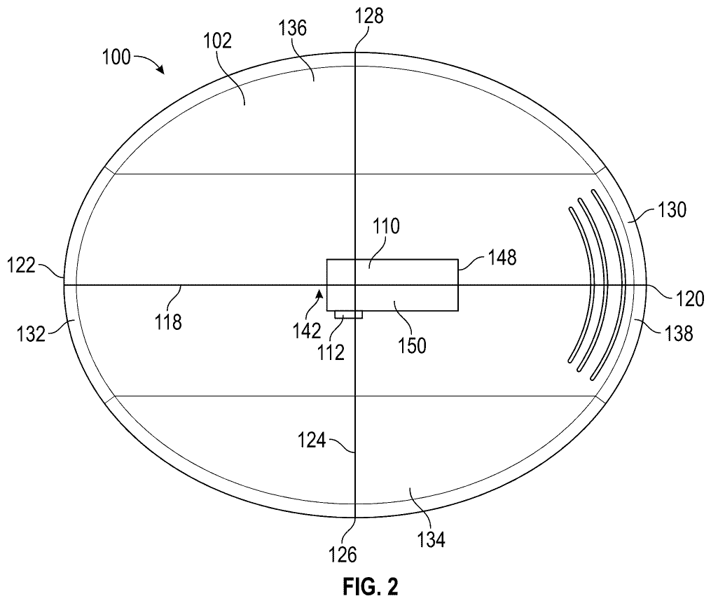

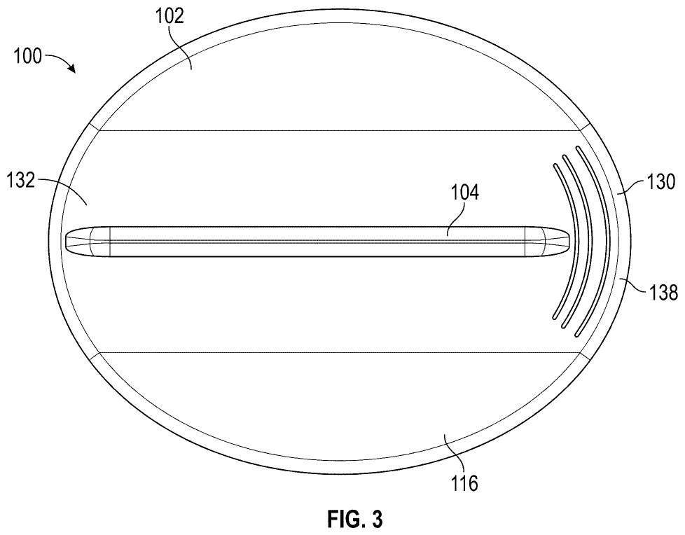

Each of FIGS. 1, 2, and 3 illustrates a first example massager 100. The massager 100 has a base 102, an elongate massage member 104, a motor 106, an extension 108, a grip 110, and a switch 112. In the illustrated embodiment, the base 102 is ovular shaped; however, the base 102 can be any desired shape including, but not limited to circular, rectangular, or the like. The base 102 has a first surface 114 and a second surface 116. The first surface 114 of the base 102 is opposite the second surface 116 of the base 102. Further, the base 102 has a length 118 that extends from a first end 120 of the base 102 to a second end 122 of the base 102. The length 118 can be any desired length 118; however, acceptable lengths 118 can include, but are not limited to, from approximately 6.985 cm (2.75 in) to approximately 15.24 cm (6 in). The base 102 also has a width 124 that extends from a third end 126 of the base 102 to a fourth end 128 of the base 102. The width 124 can be any desired width 124; however, acceptable widths 124 can include, but are not limited to, from approximately 5.715 cm (2.25 in) to approximately 12.7 cm (5 in.) It can be beneficial to have a base 102 with the length 118 being approximately 6.985 cm (2.75 in) and the width 124 being approximately 8.89 cm (3.5 in.) Further, it can also be beneficial to have a base 102 that has the length 118 being approximately 11.43 cm (4.5 in) and the width 124 being approximately 12.7 cm (5 in.) These dimensions of the base 102 can be seen as beneficial at least because it allows the massager 100 to adequately pinpoint a location of a desired area to massage on an individual. The base 102 has a first side 130, a second side 132, a third side 134, and a fourth side 136. The first side 130 is opposite the second side 132, and the third side 134 is opposite the fourth side 136. The base 102 can be created with any desired flexible material including, but not limited to, polymers. The base 102 being created out of a flexible material allows the base 102 to flex in a manner that allows pressure to be applied to an individual while also forming to the shape of an individual's body. The entirety of the base 102 can be concave.

Further, the base 102 has a concave wing 138 that defines the first side 130. The concave wing 138 does not need to define the first side 130. The concave wing 138 could alternatively define the second side 132. Also, the base 102 can have a plurality of concave wings 138 that define the first side 130 and the second side 132. Further, the concave wing 138 extends in a direction opposite to the first surface 114. The concave wing 138 can extend in the direction opposite to the first surface 114 at an angle 140. For example, the concave wing 138 can have, but is not limited to, an angle 140 of approximately 1° to approximately 90°. The concave wing 138 can be the same material as the entire base 102, or in the alternative, the concave wing 138 of the base 102 can be made of a material being more or less flexible than the remainder of the base 102. It can be seen as beneficial to have the concave wing 138 be more flexible than the remainder of the base 102 to allow it to better form to an individual's body. It can also be seen as beneficial to have the concave wing 138 be less flexible than the remainder of the base 102 to allow more pressure to be applied to an individual by the concave wing 138. Although this embodiment is described as having wings, the embodiment does not have to have visual or physical wings. If included, as detailed above, wings can have an angle as small as about 1°. Alternatively, the base can comprise a flat or substantially flat surface.

The illustrated embodiment further includes an elongate massage member 104. Although the illustrated embodiment only shows one elongate massage member 104, the massager 100 can have any number of elongate massage members 104 such as, but not limited to, one, at least one, two, at least two, a plurality, more than two, three, at least three, more than three, four, five, six, seven, eight, nine, ten, and more than ten. The elongate massage member 104 connects to the second surface 116 of the base 102. The elongate massage member 104 can be connected to the second surface 116 of the base 102 by any means such as, but not limited to, an adhesive. Alternatively, the elongate massage member can be integrally formed by the base. The elongate massage member 104 can extend in a direction away from the second surface 116 of the base 102. In the illustrated embodiment, the elongate massage member 104 extends from the first side 130 of the base 102 to the second side 132 of the base 102. The elongate massage member 104 alternatively could extend along the width 124 of the base 102 from the third end 126 to the fourth end 128. Also, the elongate massage member 104 could extend from the first end 120 to the second end 122. The elongate massage member 104 can be made from any desired material. However, it can be seen as beneficial to have the elongate massage member 104 made from a metallic material because metallic materials have a relatively low coefficient of friction when applied to the skin of an individual. Thus, allowing the massager 100 to easily glide across the skin of an individual. Further, the elongate massage member 104 can be tubular shaped; however, the elongate massage member 104 can have any desired shape so long as it allows the massager 100 to be moved across an human or animal skin in response to an unassisted force applied to the massager by a human. Also alternatively, the elongate massage members 104 can be liquid filled pads. For example, the elongate massage members 104 can comprise gel-filled pads.

In the illustrated embodiment, the motor 106 is disposed in the second side 132 of the base 102. The motor 106 can be disposed in or on any desired portion of the base 102 including, but not limited to, the concave wing 138, a central portion 142 of the base 102, the extension 108, or the grip 110. The motor 106 is adapted to produce vibration that is transferred through the base 102 to the elongate massage member 104. The motor 106 can have any desired number of speeds. However, the illustrated embodiment has only one speed that is approximately 2,000 rpm. The only one speed could be any desired speed including, but not limited to, approximately 500 rpm, 1,000 rpm, 3,000 rpm, 4,000 rpm, 5,000 rpm, 6,000 rpm, 7,000 rpm, and 9,000 rpm. Further, the massager 100 could have any desired number of motors 106 including, but not limited to, two, three, or four.

The illustrated embodiment further includes the extension 108. The extension 108 has an extension first end 144 and an extension second end 146. The extension first end 144 extends from the first surface 114 of the base 102 in a direction opposite the second surface 116 of the base 102. Further, the grip 110 is positioned at the extension second end 146. In the illustrated embodiment, the grip 110 is offset in relation to the extension 108 to allow a larger grip 110 for an individual to be able to easily hold the massager 100. However, the grip 110 can be any desired type of grip 110 that assists an individual in holding the massager 100. In alternative embodiments, the extension 108 can be eliminated, and the grip 110 can be attached or be integrally formed to the first surface 114 of the base 102. Further, the switch 112 is disposed partially in a grip first side 148 of the grip 110. The switch 112 can be positioned on any desired side 148 of the grip 110 or a grip top surface 150. The switch 112 has an on and an off position. The switch 112 is configured to turn the motor 106 on when in the on position and to turn the motor 106 off when in the off position. The switch 112 does not have to be on the grip 110; however, it is advantageous for the switch 112 to be on the grip 110 because it allows an individual to easily toggle the switch 112 between the on and off positions. Alternatively, the switch 112 could be disposed in the extension 108 or the base 102 of the massager 100.

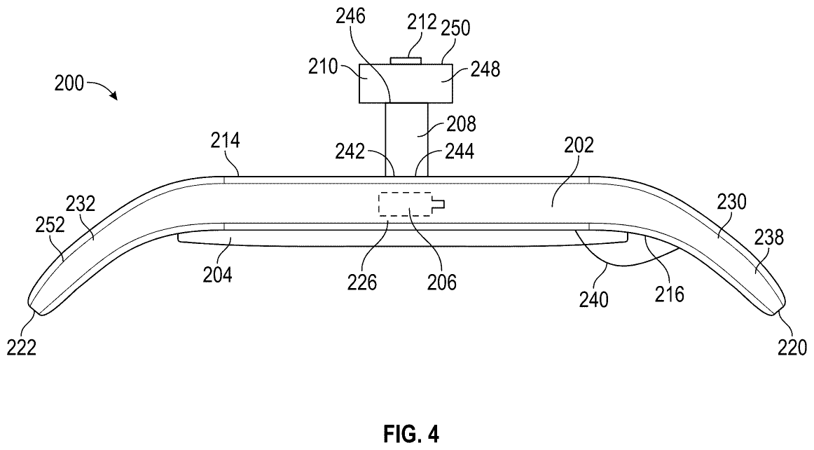

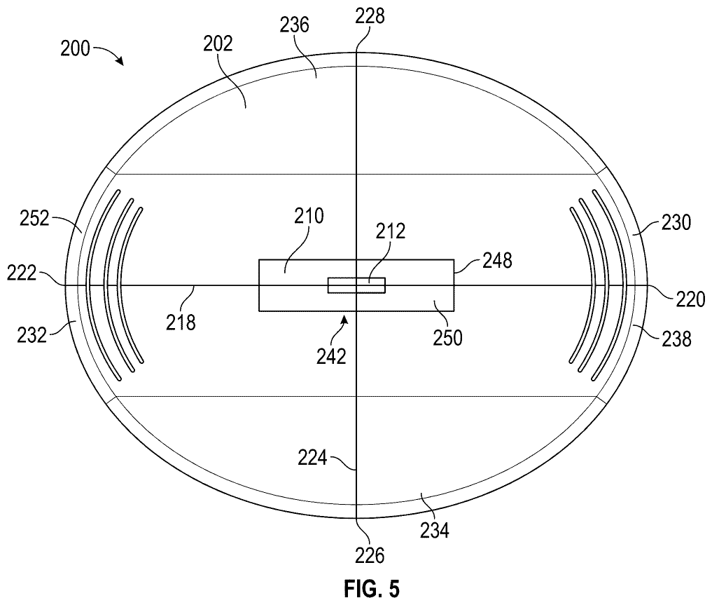

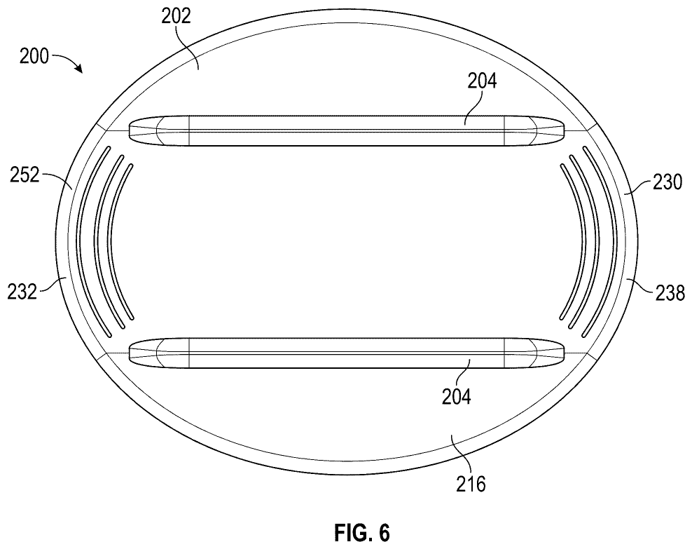

Each of FIGS. 4, 5, and 6 illustrates a second example massager 200. The massager 200 has a base 202, a plurality of elongate massage members 204, a motor 206, an extension 208, a grip 210, and a switch 212. In the illustrated embodiment, the base 202 is ovular shaped; however, the base 202 can be any desired shape including, but not limited to, circular, rectangular, or the like. The base 202 has a first surface 214 and a second surface 216. The first surface 214 of the base 202 is opposite the second surface 216 of the base 202. Further, the base 202 has a length 218 that extends from a first end 220 of the base 202 to a second end 222 of the base 202. The length 218 can be any desired length 218; however, acceptable lengths 216 can include, but are not limited to, from approximately 6.985 cm (2.75 in) to approximately 15.24 cm (6 in.) The base 202 also has a width 224 that extends from a third end 226 of the base 202 to a fourth end 228 of the base 202. The width 224 can be any desired width 224; however, acceptable widths 224 can include, but are not limited to, from approximately 5.715 cm (2.25 in) to approximately 12.7 cm (5 in.) It can be beneficial to have a base 202 with the length 218 being approximately 6.985 cm (2.75 in) and the width 224 being approximately 8.89 cm (3.5 in.) Further, it can also be beneficial to have a base 202 that has the length 218 being approximately 11.43 cm (4.5 in) and the width 224 being approximately 12.7 cm (5 in.) These dimensions of the base 202 can be seen as beneficial at least because it allows the massager 200 to adequately pinpoint a location of a desired area to massage on an individual. The base 202 further has a first side 230, a second side 232, a third side 234, and a fourth side 236. The first side 230 is opposite the second side 232, and the third side 234 is opposite the fourth side 236. The base 202 can be created with any desired flexible material including, but not limited to, polymers. The base 202 being created out of a flexible material allows the base 202 to flex in a manner that allows pressure to be applied to an individual while also forming to the shape of an individual's body. The entirety of the base 202 can be concave.

Further, the base 202 has a first concave wing 238 and a second concave wing 252. The first concave wing 238 defines the first side 230 of the base 202, and the second concave wing 252 defines the second side 232 of the base 202. The massager 200 does not have to have the first concave wing 238 and the second concave wing 252. The massager 200 could have only one concave wing, such as in the first example massager 200. Further, the first concave wing 238 and the second concave wing 252 extends in a direction opposite to the first surface 214. The first concave wing 238 and the second concave wing 252 can extend in the direction opposite to the first surface 214 at an angle 240. For example, the first concave wing 238 and the second concave wing 252 can have, but is not limited to, an angle 240 of approximately 1° to approximately 90°. The first concave wing 238 and the second concave wing 252 can be the same material as the remainder of the base 202, or in the alternative, the first concave wing 238 and the second concave wing 252 of the base 202 can be made of a material being more or less flexible than the remainder of the base 202. It can be seen as beneficial to have the first concave wing 238 and the second concave wing 252 be more flexible than the remainder of the base 202 to allow the massager 200 to better form to an individual's body. It can also be seen as beneficial to have the first concave wing 238 and the second concave wing 252 be less flexible than the remainder of the base 202 to allow more pressure to be applied to an individual by the first concave wing 238 and the second concave wing 252. Although this embodiment is described as having wings, the embodiment does not have to have visual or physical wings. If included, as detailed above, wings can have an angle as small as about 1°. Alternatively, the base can comprise a flat or substantially flat surface.

The illustrated embodiment further has the plurality of elongate massage members 204. Although the illustrated embodiment only shows two elongate massage members 204, the massager 200 can have any number of elongate massage members 204 such as, but not limited to, one, three, five, six, and nine. The plurality of elongate massage members 204 connect to the second surface 216 of the base 202. The plurality of elongate massage members 204 can be connected to the second surface 216 of the base 202 by any means such as, but not limited to, an adhesive or integrally formed with the base 202. The plurality of elongate massage members 204 can extend in a direction away from the second surface 216 of the base 202. In the illustrated embodiment, the plurality of elongate massage members 204 extend from the first side 230 of the base 202 to the second side 232 of the base 202. The plurality of elongate massage members 204 alternatively could extend along the width 224 of the base 202 from the third end 226 of the base 202 to the fourth end 228 of the base 202. Also, the plurality of elongate massage members 204 could extend from the first end 220 to the second end 222. The plurality of elongate massage members 204 can be made from any desired material. However, it can be seen as beneficial to have the plurality of elongate massage members 204 made from a metallic material because metallic materials have a relatively low coefficient of friction when applied to the skin of an individual. Thus, allowing the massager 200 to easily glide across the skin of an individual. Further, the plurality of elongate massage members 204 can be tubular shaped; however, the plurality of elongate massage members 204 can have any desired shape so long as it allows the massager 200 to glide across an individual's skin. Additionally, although the plurality of elongate massage members 204 appear to have similar lengths, the plurality of elongate massage members 204 can be a variety of different shapes. The plurality of the elongate massage members 204 also do not have to be positioned anywhere in particular on the second surface 216 on the base 202. For example, the plurality of elongate massage members 204 can be closer or farther together than as illustrated. Also alternatively, the elongate massage members 204 can be liquid filled pads. For example, the elongate massage members 204 can comprise gel-filled pads.

In the illustrated embodiment, the motor 206 is disposed in a central portion 242 of the base 202. The motor 206 can be disposed in or on any desired portion of the base 202 including, but not limited to, the first concave wing 238, the second concave wing 252, the extension 208, or the grip 210. The motor 206 is adapted to produce a vibration that is transferred through the base 202 to the plurality of elongate massage members 204. The motor 206 can have any desired number of speeds. However, the motor 206 in the illustrated embodiment has three speeds that include approximately 2,000 rpm, 4,000 rpm, and 6,000 rpm. It can be seen that these speeds are beneficial at least because they provide an optimal amount of vibration through the plurality of elongate massage members 204. The speeds could alternatively include, but are not limited to, approximately 500 rpm, 1,000 rpm, 3,000 rpm, 5,000 rpm, 7,000 rpm, and 9,000 rpm. Further, the massager 200 could have any desired number of motors 206 including, but not limited to, two, three, or four.

The illustrated embodiment further includes the extension 208. The extension 208 has an extension first end 244 and an extension second end 246. The extension first end 244 extends from the first surface 214 of the base 202 in a direction opposite the second surface 216 of the base 202. Further, the grip 210 is positioned at the extension second end 246. In the illustrated embodiment, the grip 210 is centered in relation to the extension 208 to allow an individual to be able to easily hold the massager 200. However, the grip 210 can be any desired type of grip 210 that assists an individual in being able to hold the massager 200. In alternative embodiments, the extension 208 can be eliminated, and the grip 210 can be attached or be integrally formed to the first surface 214 of the base 202. Further, the switch 212 is disposed partially in a grip top surface 250 of the grip 210. The switch 212 can positioned on any grip side 248 of the grip 210. The switch 212 has an on and an off position. The switch 212 is configured to turn the motor 206 on when in the on position and to turn the motor 206 off when in the off position. If the on position on the switch 212 is pushed after it has turned on, the speed of the motor 206 will change, hence changing the amount of vibration that is produced. The switch 212 does not have to be on the grip 210; however, it is advantageous for the switch 212 to be on the grip 210 because it allows an individual to easily toggle the switch 212 between the on and off positions.

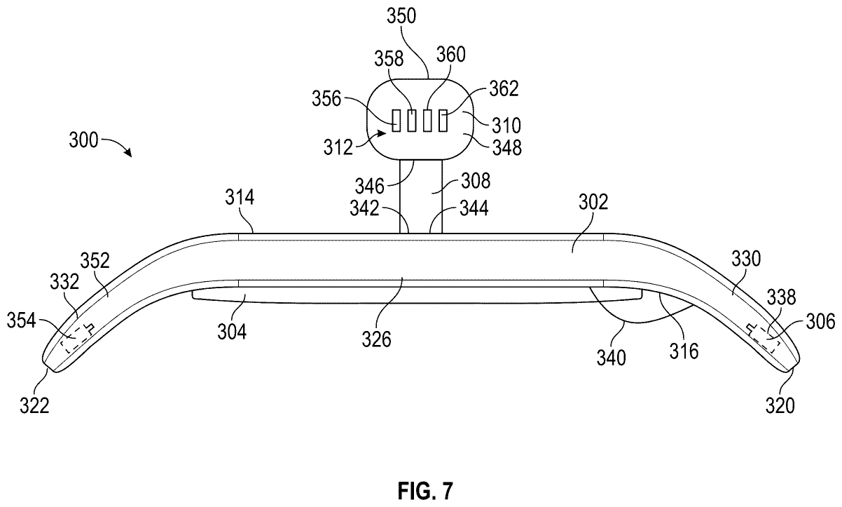

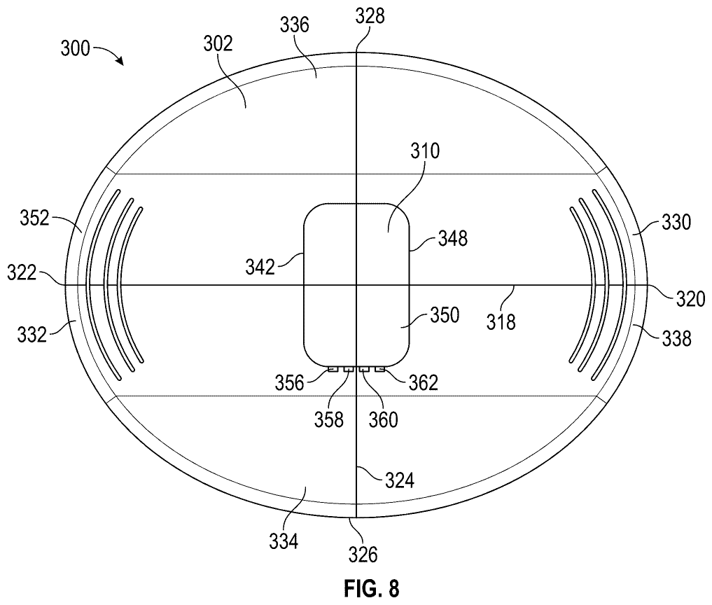

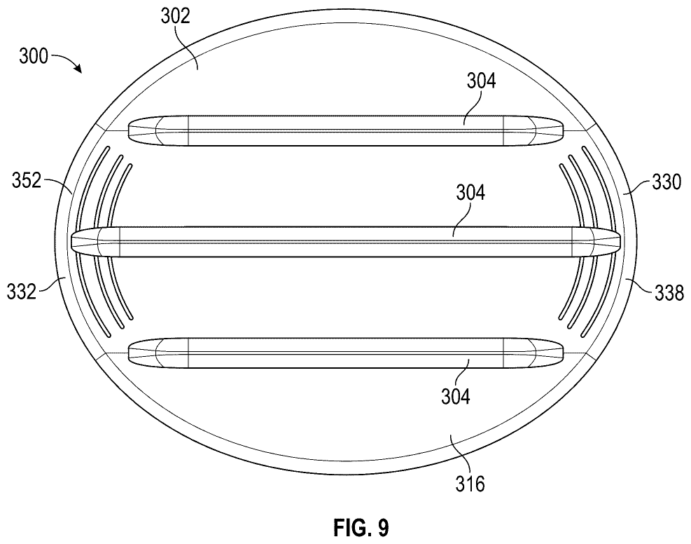

Each of FIGS. 7, 8, and 9 illustrates a third example massager 300. The massager 300 has a base 302, a plurality of elongate massage members 304, a first motor 306, a second motor 354, an extension 308, a grip 310, and a switch 312. In the illustrated embodiment, the base 302 is ovular shaped; however, the base 302 can be any desired shape including, but not limited to, circular, rectangular, or the like. The base 302 has a first surface 314 and a second surface 316. The first surface 314 of the base 302 is opposite the second surface 316 of the base 302. Further, the base 302 has a length 318 that extends from a first end 320 of the base 302 to a second end 322 of the base 302. The length 318 can be any desired length 318; however, acceptable lengths 318 can include, but are not limited to, from approximately 6.985 cm (2.75 in) to approximately 15.24 cm (6 in.) The base 302 also has a width 324 that extends from a third end 326 of the base 302 to a fourth end 328 of the base 302. The width 324 can be any desired width 324; however, acceptable widths 324 can include, but are not limited to, from approximately 5.715 cm (2.25 in) to approximately 12.7 cm (5 in.) It can be beneficial to have a base 302 with the length 318 being approximately 6.985 cm (2.75 in) and the width 324 being approximately 8.89 cm (3.5 in.) Further, it can also be beneficial to have a base 302 that has the length 318 being approximately 11.43 cm (4.5 in) and the width 324 being approximately 12.7 cm (5 in.) These dimensions of the base 302 can be seen as beneficial at least because it allows the massager 300 to adequately pinpoint a location of a desired area to massage on an individual. The base 302 further has a first side 330, a second side 332, a third side 334, and a fourth side 336. The first side 330 is opposite the second side 332, and the third side 334 is opposite the fourth side 336. The base 302 can be created with any desired flexible material including, but not limited to, polymers. The base 302 being created out of a flexible material allows the base 302 to flex in a manner that allows pressure to be applied to an individual while also forming to the shape of an individual's body. The entirety of the base 302 can be concave.

Further, the base 302 has a first concave wing 338 and a second concave wing 352. The first concave wing 338 defines the first side 330 of the base 302, and the second concave wing 352 defines the second side 332 of the base 302. The massager 300 does not have to have a first concave wing 338 and a second concave wing 352. The massager 300 could have only one concave wing, such as in the first example massager 300. Further, the first concave wing 338 and the second concave wing 352 extend in a direction opposite to the first surface 314. The first concave wing 338 and the second concave wing 352 can extend in the direction opposite to the first surface 314 at an angle 340. For example, the first concave wing 338 and the second concave wing 352 can have, but is not limited to, an angle 340 of approximately 1° to approximately 90°. The first concave wing 338 and the second concave wing 352 can be the same material as the remainder of the base 302, or in the alternative, the first concave wing 338 and the second concave wing 352 portions of the base 302 can be made of a material being more or less flexible than the other portions of the base 302. It can be seen as beneficial to have the first concave wing 338 and the second concave wing 352 be more flexible than the remainder of the base 302 to allow the massager 300 to better form to an individual's body. It can also be seen as beneficial to have the first concave wing 338 and the second concave wing 352 be less flexible than the remainder of the base 302 to allow more pressure to be applied to an individual by the first concave wing 338 and the second concave wing 352. Although this embodiment is described as having wings, the embodiment does not have to have visual or physical wings. If included, as detailed above, wings can have an angle as small as about 1°. Alternatively, the base can comprise a flat or substantially flat surface.

The illustrated embodiment further has the plurality of elongate massage members 304. Although the illustrated embodiment only shows three elongate massage members 304, the massager 300 can have any number of elongate massage members 304 such as, but not limited to, one, two, four, five, six, and nine. The plurality of elongate massage members 304 connect to the second surface 316 of the base 302. The plurality of elongate massage members 304 can be connected to the second surface 316 of the base 302 by any means such as, but not limited to, an adhesive or be integrally formed with the base 302. The plurality of elongate massage members 304 can extend in a direction away from the second surface 316 of the base 302. In the illustrated embodiment, the plurality of elongate massage members 304 extend from the first side 330 of the base 302 to the second side 332 of the base 302. The plurality of elongate massage members 304 alternatively could extend along the width 324 of the base 302 from the third end 326 of the base 302 to the fourth end 328 of the base 302. Also, the plurality of elongate massage members 304 could extend from the first end 320 to the second end 322. The plurality of elongate massage members 304 can be made from any desired material. However, it can be seen as beneficial to have the plurality of elongate massage members 304 made from a metallic material because metallic materials have a relatively low coefficient of friction when applied to the skin of an individual. Thus, allowing the massager 300 to easily glide across the skin of an individual. Further, the plurality of elongate massage members 304 can be tubular shaped; however, the plurality of elongate massage members 304 can have any desired shape so long as it allows the massager 300 to glide across an individual's skin. In the illustrated embodiment, the plurality of elongate massage members 304 have a variety of different lengths. The most central elongate massage member is longer than the other elongate massage members 304. Although the plurality of elongate massage members 304 have different lengths, the plurality of elongate massage members 304 can be of the same length. The plurality of the elongate massage members 304 also do not have to be positioned anywhere in particular on the second surface 316 on the base 302. For example, the plurality of elongate massage members 304 can be closer together or farther apart than as illustrated. Also alternatively, the elongate massage members 304 can be liquid filled pads. For example, the elongate massage members 304 can comprise gel-filled pads.

The illustrated embodiment has the first motor 306 disposed in the first concave wing 338 and the second motor 354 disposed in the second concave wing 352. The first motor 306 and the second motor 354 can be disposed in or on any desired portion of the base 302 including, but not limited to, the central portion 342 of the base 302, the extension 308, or the grip 310. The first motor 306 and the second motor 354 are adapted to produce a vibration that is transferred through the base 302 to the plurality of elongate massage members 304. In the illustrated embodiment, the first motor 306 and the second motor 354 have different speeds. The first motor 306 has a first speed, and the second motor 354 has a second speed. The first speed being less than the second speed. The first motor 306 and the second motor 354 can have any desired number of speeds. Each of the first motor 306 and the second motor 354 has a different speed. The first motor 306 has a speed of approximately 2,000 rpm and the second motor 354 has a speed of approximately 4,000 rpm. When both the first motor 306 and the second motor 354 are powered on, there is a total of approximately 6,000 rpm between the first motor 306 and the second motor 354. It can be seen that these speeds are beneficial at least because they provide an optimal amount of vibration through the plurality of elongate massage members 304. The speeds could alternatively include, but are not limited to, approximately 500 ppm, 1,000 rpm, 3,000 rpm, 5,000 rpm, 7,000 rpm, 9,000 rpm. Further, the massager 300 could have any desired number of motors including, but not limited to, one, three, or four.

The illustrated embodiment further includes the extension 308. The extension 308 has an extension first end and an extension second end 346. The extension first end extends from the first surface 314 of the base 302 in a direction opposite the second surface 316 of the base 302. Further, the grip 310 is positioned at the extension second end 346. In the illustrated embodiment, the grip 310 is centered in relation to the extension 308 to allow an individual to be able to easily hold the massager 300. However, the grip 310 can be any desired type of grip 310 that assists an individual in holding the massager 300. In alternative embodiments, the extension 308 can be eliminated, and the grip 310 can be attached or be integrally formed to the first surface 314 of the base 302. Further, the switch 312 is disposed partially in a grip side 348 of the grip 310. The switch 312 can be positioned on any desired side 348 of the grip 310 or the grip top surface 350. The switch 312 includes a first button 356, a second button 358, a third button 360, and a fourth button 362. The first button 356 is configured to turn the first motor 306 and the second motor 354 off by putting the switch 312 in an off position. The second button 358 is configured to turn the first motor 306 on by putting the switch 312 in an on position. The third button 360 is configured to turn the second motor 354 on by putting the switch 312 in an on position. The fourth button 362 is configured to turn the first motor 306 and the second motor 354 on by putting the switch 312 in an on position. The switch 312 is configured to turn the first motor 306 and second motor 354 on when in the respective on positions and to turn the first motor 306 and second motor 354 off when the switch 312 is in an off position. The switch 312 does not have to be on the grip 310; however, it is advantageous for the switch 312 to be on the grip 310 because it allows an individual to easily toggle the switch 312 between the on and off positions.

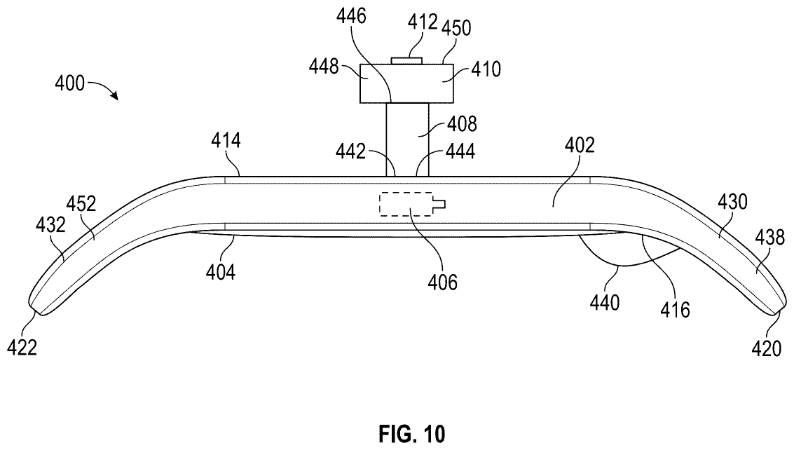

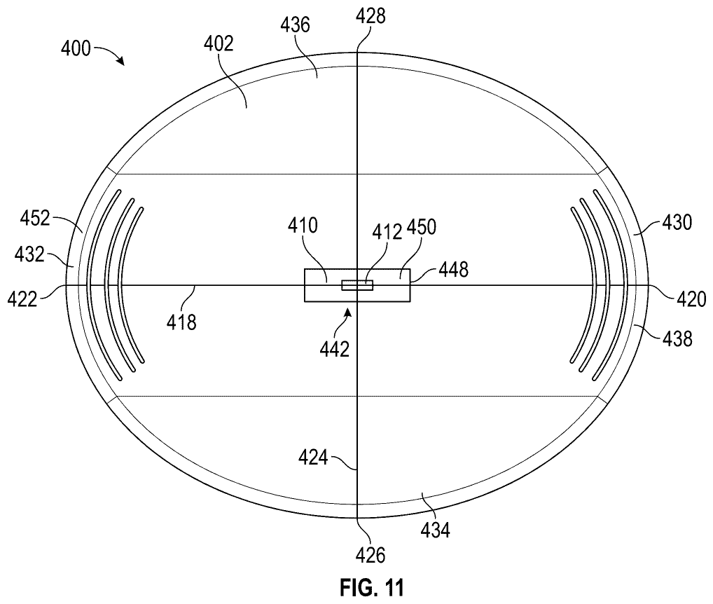

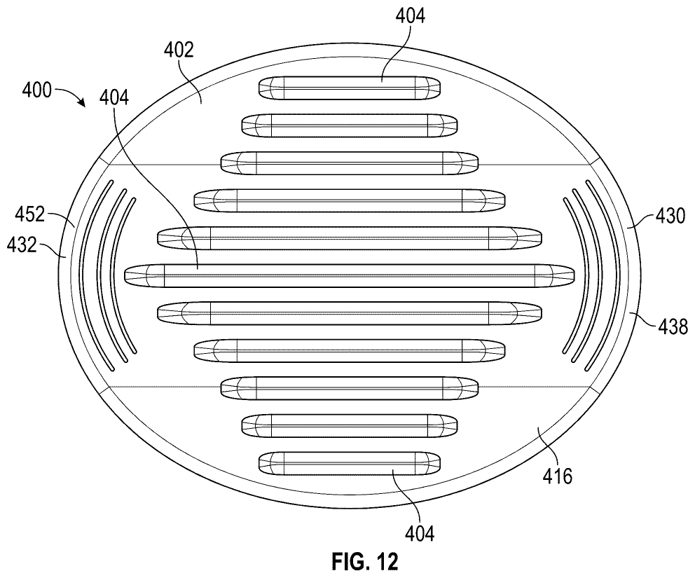

Each of FIGS. 10, 11, and 12 illustrates a fourth example massager 400. The massager 400 has a base 402, a plurality of elongate massage members 404, a motor 406, an extension 408, a grip 410, and a switch 412. In the illustrated embodiment, the base 402 is circular shaped; however, the base 402 can be any desired shape including, but not limited to, ovular, rectangular, or the like. The base 402 has a first surface 414 and a second surface 416. The first surface 414 of the base 402 is opposite the second surface 416 of the base 402. Further, the base 402 has a length 418 that extends from a first end 420 of the base 402 to a second end 422 of the base 402. The base 402 also has a width 424 that extends from a third end 426 of the base 402 to a fourth end 428 of the base 402. In the illustrated embodiment, the width 424 of the base 402 and the length 418 of the base 402 are the same. Acceptable lengths 418 and widths 424 can include, but are not limited to, from approximately 5.715 cm (2.25 in) to approximately 15.24 cm (6 in.) It can be seen as being advantageous to have the length 418 and width 424 of the base 402 being approximately 7 cm (2.75 in), 8.89 cm (3.5 in), 11.43 cm (4.5 in), or 12.7 cm (5 in.) These dimensions of the base 402 can be seen as beneficial at least because it allows the massager 400 to adequately pinpoint a location of a desired area to massage on an individual. The base 402 further has a first side 430, a second side 432, a third side 434, and a fourth side 436. The first side 430 is opposite the second side 432, and the third side 434 is opposite the fourth side 436. The base 402 can be created with any desired flexible material including, but not limited to, polymers. The base 402 being created out of a flexible material allows the base 402 to flex in a manner that allows pressure to be applied to an individual while also forming to the shape of an individual's body. The entirety of the base 402 can be concave.

Further, the base 402 has a first concave wing 438 and a second concave wing 452. The first concave wing 438 defines the first side 430 of the base 402, and the second concave wing 452 defines the second side 432 of the base 402. The massager 400 does not have to have the first concave wing 438 and the second concave wing 452. The massager 400 could have only one concave wing, such as in the first example massager 400. Further, the first concave wing 438 and the second concave wing 452 can extend in the direction opposite to the first surface 414 at an angle 440. For example, the first concave wing 438 and the second concave wing 452 can have, but is not limited to, an angle 440 of approximately 1° to approximately 90°. The first concave wing 438 and the second concave wing 452 can be the same material as the remainder of the base 402, or in the alternative, the first concave wing 438 and the second concave wing 452 portions of the base 402 can be made of a material being more or less flexible than the other portions of the base 402. It can be seen as beneficial to have the first concave wing 438 and the second concave wing 452 be more flexible than the remainder of the base 402 to allow the massager 400 to better form to an individual's body. It can also be seen as beneficial to have the first concave wing 438 and the second concave wing 452 be less flexible than the remainder of the base 402 to allow more pressure to be applied to an individual by the first concave wing 438 and the second concave wing 452. Although this embodiment is described as having wings, the embodiment does not have to have visual or physical wings. If included, as detailed above, wings can have an angle as small as about 1°. Alternatively, the base can comprise a flat or substantially flat surface.

The illustrated embodiment further has the plurality of elongate massage members 404. Although the illustrated embodiment shows eleven elongate massage members 404, the massager 400 can have any number of elongate massage members 404 such as, but not limited to, one, two, three, four, five, six, and nine. The plurality of elongate massage members 404 are partially disposed in the base 402. The plurality of elongate massage members 404 can also be connected to the second surface 416 of the base 402 by any means such as, but not limited to, an adhesive or be integrally formed with the base 402. The plurality of elongate massage members 404 can extend in a direction away from the second surface 416 of the base 402. In the illustrated embodiment, the plurality of elongate massage members 404 extend from the first side 430 of the base 402 to the second side 432 of the base 402. The plurality of elongate massage members 404 alternatively could extend along the width 424 of the base 402 from the third end 426 of the base 402 to the fourth end 428 of the base 402. Also, the plurality of elongate massage members 404 could extend from the first end 420 to the second end 422. The plurality of elongate massage members 404 can be made from any desired material. However, it can be seen as beneficial to have the plurality of elongate massage members 404 made from a metallic material because metallic materials have a relatively low coefficient of friction when applied to the skin of an individual. Thus, allowing the massager 400 to easily glide across the skin of an individual. Further, the plurality of elongate massage members 404 can be tubular shaped; however, the plurality of elongate massage members 404 can have any desired shape so long as it allows the massager 400 to glide across an individual's skin. Additionally, although the plurality of elongate massage members 404 appear to have a variety of different lengths, the plurality of elongate massage members 404 can have similar lengths. The plurality of the elongate massage members 404 also do not have to be positioned anywhere in particular on the second surface 416 on the base 402. For example, the plurality of elongate massage members 404 can be closer or farther together than as illustrated. Also alternatively, the elongate massage members 204 can be liquid filled pads. For example, the elongate massage members 204 can comprise gel-filled pads.

In the illustrated embodiment, the motor 406 is disposed in a central portion 442 of the base 402. The motor 406 can be disposed in or on any desired portion of the base 402 including, but not limited to, the first concave wing 438, the second concave wing 452, the extension 408, or the grip 410. The motor 406 is adapted to produce a vibration that is transferred through the base 402 to the plurality of elongate massage members 404. The motor 406 can have any desired number of speeds. However, the illustrated embodiment has only one speed that is approximately 4,000 rpm. It can be seen that this speed is beneficial at least because it can provide an optimal amount of vibration through the plurality of elongate massage members 404. The speeds could alternatively include, but are not limited to, approximately 500 rpm, 1,000 rpm, 3,000 rpm, 5,000 rpm, 7,000 rpm, and 9,000 rpm.

The illustrated embodiment further includes the extension 408. The extension 408 has an extension first end 444 and an extension second end 446. The extension first end 444 extends from the first surface 414 of the base 402 in a direction opposite the second surface 416 of the base 402. Further, the grip 410 is positioned at the extension second end 446. In the illustrated embodiment, the grip 410 is centered in relation to the extension 408 to allow an individual to be able to easily hold the massager 400. However, the grip 410 can be any desired type of grip 410 that assists an individual in being able to hold the massager 400. In alternative embodiments, the extension 408 can be eliminated, and the grip 410 can be attached or be integrally formed to the first surface 414 of the base 402. Further, the switch 412 is disposed partially in a grip top surface 450 of the grip 410. The switch 412 can positioned on any grip side 448 of the grip 410. The switch 412 has an on and an off position. The switch 412 is configured to turn the motor 406 on when in the on position and to turn the motor 406 off when in the off position. The switch 412 does not have to be on the grip 410; however, it is advantageous for the switch 412 to be on the grip 410 because it allows an individual to easily toggle the switch 412 between the on and off positions.

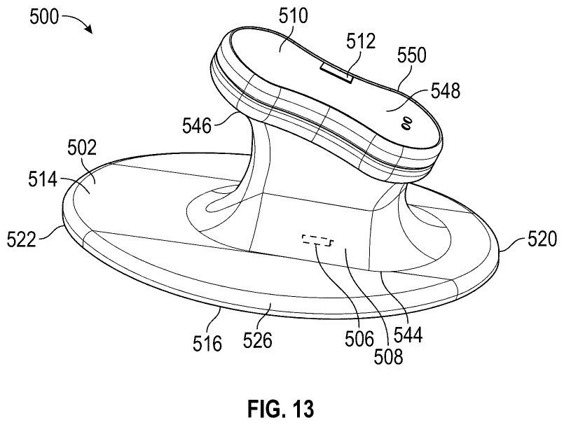

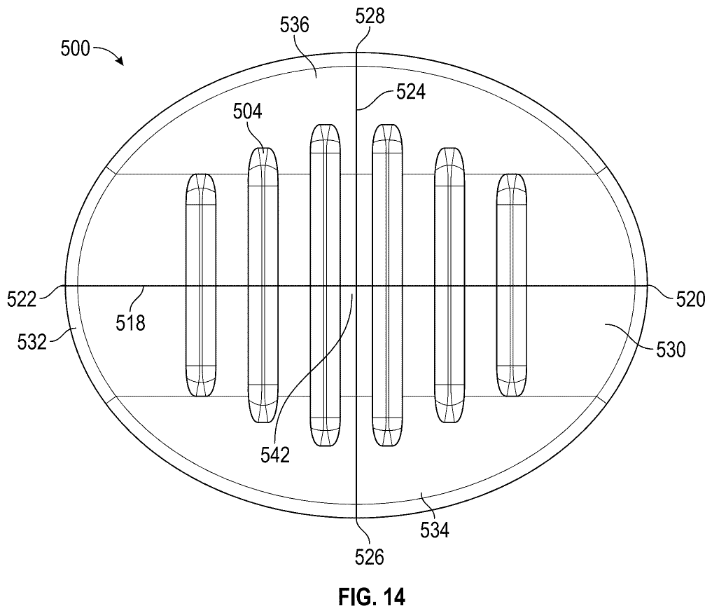

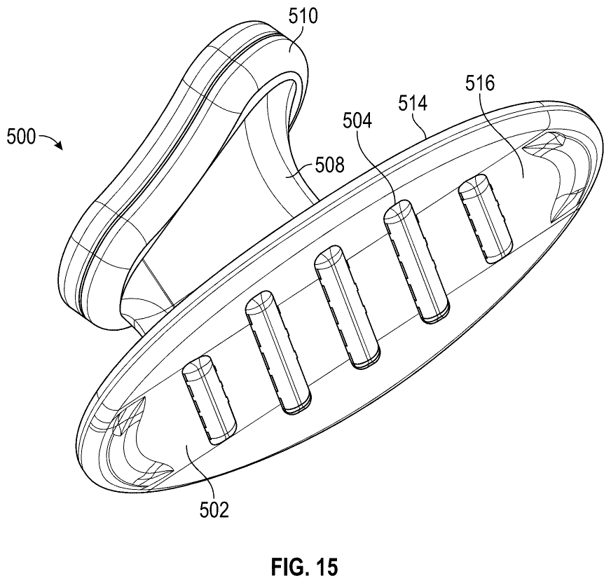

Each of FIGS. 13, 14, and 15 illustrates a fifth example massager 500. The massager 500 has a base 502, an elongate massage member 504, a motor 506, an extension 508, a grip 510, and a switch 512. In the illustrated embodiment, the base 502 is ovular shaped; however, the base 502 can be any desired shape including, but not limited to circular, rectangular, or the like. The base has a first surface 514 and a second surface 516. The first surface 514 of the base 502 is opposite the second surface 516 of the base 502. Further, the base 502 has a length 518 that extends from a first end 520 of the base 502 to a second end 522 of the base 502. The length 518 can be any desired length; however, acceptable lengths can include, but are not limited to, from approximately 6.985 cm (2.75 in) to approximately 15.24 cm (6 in). The base 502 also has a width 524 that extend from a third end 526 of the base 502 to a fourth end 528 of the base 502. The width 524 can be any desired width; however, acceptable widths can include, but are not limited to, from approximately 5.715 cm (2.25 in) to approximately 12.7 cm (5 in.) It can be beneficial to have a base 502 with the length 518 being approximately 6.985 cm (2.75 in) and the width 524 being approximately 8.89 cm (3.5 in.) Further, it can also be beneficial to have a base 502 that has the length 518 being approximately 11.43 cm (4.5 in) and the width 524 being approximately 12.7 cm (5 in.) These dimensions of the base 502 can be seen as beneficial at least because it allows the massager 500 to adequately pinpoint a location of a desired area to massage on an individual. The base 502 has a first side 530, a second side 532, a third side 534, and a fourth side 536. The first side 530 is opposite the second side 532, the third side 534 is opposite the fourth side 536. The base 502 can be created with any desired flexible material including, but not limited to, polymers. The base 502 being created out of a flexible material allows the base 502 to flex in a manner that allows pressure to be applied to an individual while also forming to the shape of an individual's body. The entirety of the base 502 can be concave; however, in the illustrated embodiment, the base 502 has a relatively planar second surface 516 with a slight curve to make the base 502 concave.

The illustrated embodiment further has the elongate massage member 504. Although the illustrated embodiment shows six elongate massage members 504, the massager 500 can have any number of elongate massage members 504 such as, but not limited to, one, two, three, five, and nine. The elongate massage members 504 connect to the second surface 516 of the base 502. The elongate massage members 504 can be connected to the second surface 516 of the base 502 by any means such as, but not limited to, an adhesive or be integrally formed. The elongate massage members 504 can extend in a direction away from the second surface 516 of the base 502. In the illustrated embodiment, the elongate massage members 504 are integrally formed with the base 502 and extend from the third side 534 of the base 502 to the fourth side 536 of the base 502. The elongate massage members 504 alternatively could extend along the length of the base 502 from the first end 520 to the second end 522. Also, the elongate massage members 504 could extend from the first end 520 to the second end 522. The elongate massage members 504 can be made from any desired material. However, it can be seen as beneficial to have the elongate massage member 504 made from a metallic material because metallic materials have a relatively low coefficient of friction when applied to the skin of an individual. Thus, this allows the massager 500 to glide across the skin of an individual. Further, the elongate massage members 504 can be tubular shaped; however, the elongate massage members 504 can have any desired shape so long as it allows the massager 500 to glide across an individual's skin. Also, the elongate massage members 504 could be liquid filled pads. For example, the liquid filled pads can be gel-filled pads.

In the illustrated embodiment, the motor 506 is disposed in the base 502 at a location where the extension 508 comes in contact with the base 502. The motor 506 can be disposed in or on any desired portion of the base 502 including, but not limited to, a central portion 542 of the base 502, the extension 508, or the grip 510. The motor 506 is adapted to produce vibration that is transferred through the base 502 to the elongate massage member 504. The motor 506 can have any desired number of speeds. However, the illustrated embodiment has three speeds that are approximately 2,000 rpm, 3,000 rpm, and 6,000 rpm. The speeds could alternatively include, but are not limited to, approximately 500 rpm, 1,000 rpm, 3,000 rpm, 5,000 rpm, 7,000 rpm, and 9,000 rpm. Further, the massager 500 could have any desired number of motors 506 including, but not limited to, two, three, or four.

The illustrated embodiment further includes the extension 508. The extension 508 has an extension first end 544 and an extension second end 546. The extension first end 544 extends from the first surface 514 of the base 502 in a direction opposite the second surface 516 of the base 502. Further, the grip 510 is positioned at the extension second end 546. In the illustrated embodiment, the grip 510 is offset in relation to the extension 508 to allow a larger grip 510 for an individual to be able to easily hold the massager 500. However, the grip 510 can be any desired type of grip 510 that assists an individual in holding the massager 500. In alternative embodiments, the extension 508 can be eliminated, and the grip 510 can be attached or be integrally formed to the first surface 514 of the base 502. Further, the switch 512 is disposed in a grip top surface 550 of the grip 510. The switch 512 can be positioned on any desired side of the grip 510. The switch 512 is configured to turn the motor 506 on when the switch 512 is activated. When the switch 512 is activated again, the motor 506 will cycle through the before mentioned speeds. When activating the switch 512 after cycling through all of the speeds, the motor 506 will shut off. The switch 512 does not have to be on the grip 510; however, it is advantageous for the switch 512 to be on the grip 510 because it allows an individual to easily toggle the switch 512 between the speeds. Alternatively, the switch 512 could be disposed in the extension 508 or the base 502 of the massager 500.

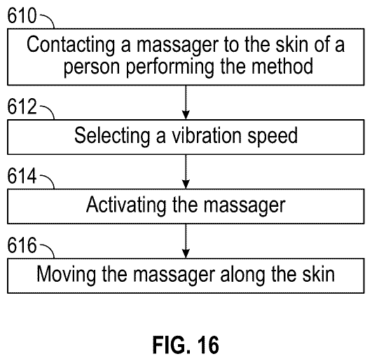

FIG. 16 illustrates a method of performing self treatment for enhancing lymphatic drainage. Alternatively, the method of performing self treatment can be for self treatment of lymphedema. An initial step 610 comprises contacting a massager according to an embodiment to the skin of the person performing the method. The skin can be skin superficial to an area presenting poor lymphatic drainage, lymphedema, or of an area that has been exposed to radiation treatment, as part of cancer treatment, for example. The massager can be a massager according to any embodiment. An optional step 612 comprises selecting a vibration speed, and can be included if the massager used in step 610 has multiple vibration speeds. Another step 614 comprises activating the massager, such as by powering on the massager. This step 614 creates vibrations on the skin contacted in step 610. Another step 616 comprises moving the massager along the skin.

This method of performing self treatment for enhancing lymphatic drainage, and the massagers described herein, can provide several benefits including, but not limited to, assisting in lymphatic drainage throughout the body; improve density to irradiated regions; reduce formation of scar tissue; address lymphedema in areas where lymph nodes have been removed as part of oncological diagnosis and drainage is lessened due to reduced nodes; treat and reduce pain resulting from mastitis; nerve training/re-education after surgery or damage to the body; improving circulation; pain reduction in areas of swelling or nerve damage; promotes a sense of wellness and wellbeing; muscle and soft tissue recovery; improved joint mobility; increased blood flow; wound healing i.e. treatment of bed sores/pressure wounds; to keep soft tissue loose and assist in absorption of skin moisturizers while receiving radiation treatment; promotion of wellness; treating and reducing pain resulting from surgery, radiation therapy, lymphedema, or other condition resulting in paint; and promotion of healing.

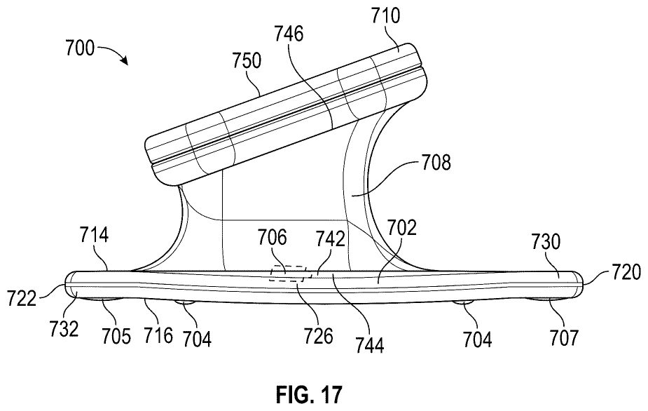

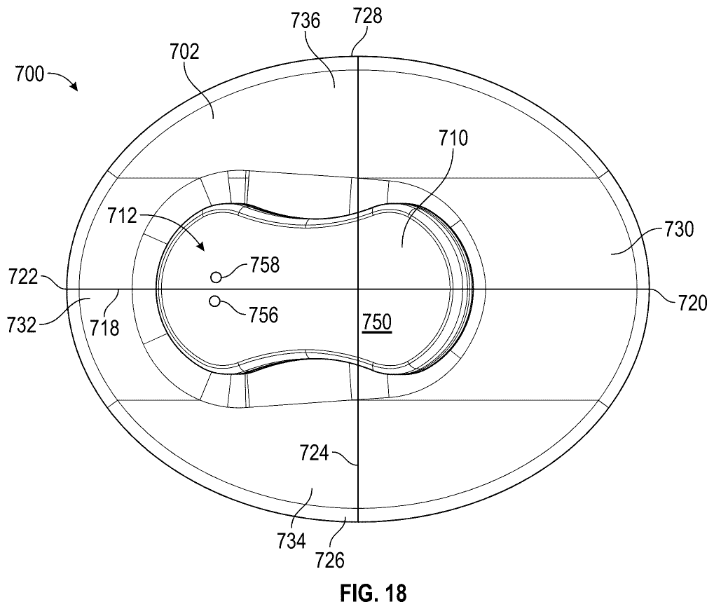

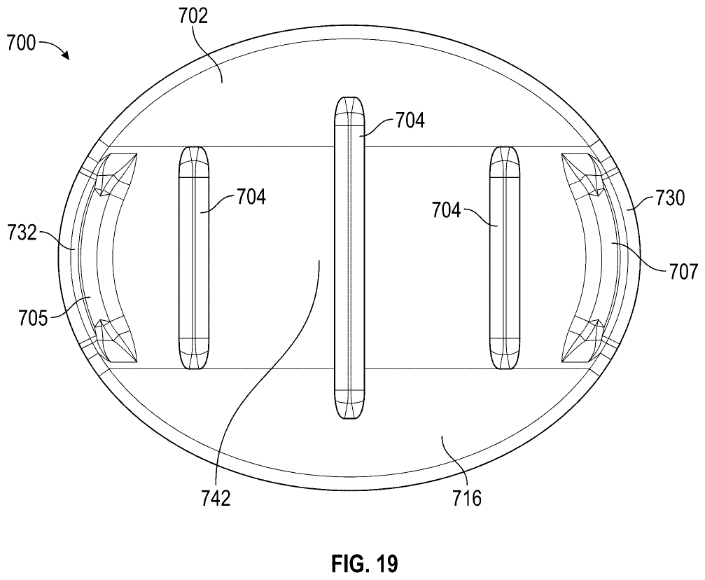

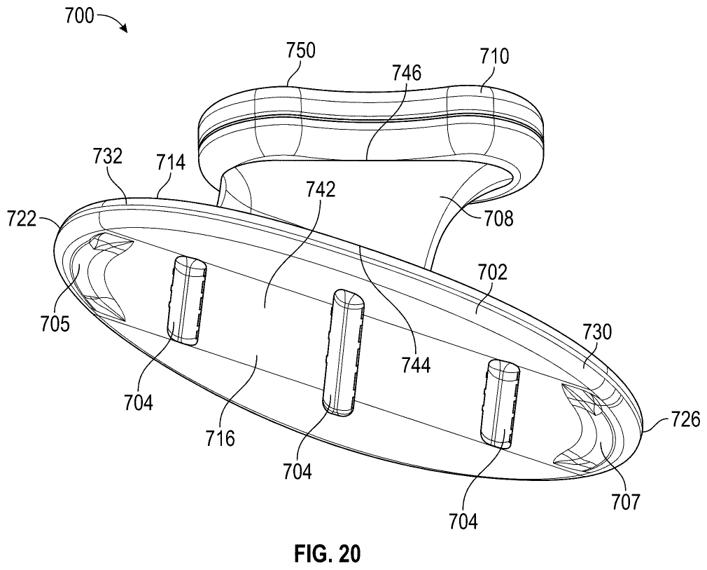

Each of FIGS. 17, 18, 19, and 20 illustrates a sixth example massager 700. The massager 700 has a base 702, an elongate massage member 704, a motor 706, an extension 708, a grip 710, and a switch 712. In the illustrated embodiment, the base 702 is ovular shaped; however, the base 702 can be any desired shape including, but not limited to circular, rectangular, or the like. The base 702 has a first surface 714 and a second surface 716. The first surface 714 of the base 702 is opposite the second surface 716 of the base 702. Further, the base 702 has a length 718 that extends from a first end 720 of the base 702 to a second end 722 of the base 702. The length 718 can be any desired length 218; however, acceptable lengths 178 can include, but are not limited to, from approximately 6.985 cm (2.75 in) to approximately 15.24 cm (6 in). The base 702 also has a width 724 that extends from a third end 726 of the base 102 to a fourth end 728 of the base 702. The width 724 can be any desired width 724; however, acceptable widths 724 can include, but are not limited to, from approximately 5.715 cm (2.25 in) to approximately 12.7 cm (5 in.) It can be beneficial to have a base 702 with the length 718 being approximately 6.985 cm (2.75 in) and the width 724 being approximately 8.89 cm (3.5 in.) Further, it can also be beneficial to have a base 702 that has the length 718 being approximately 11.43 cm (4.5 in) and the width 724 being approximately 12.7 cm (5 in.) These dimensions of the base 702 can be seen as beneficial at least because it allows the massager 700 to adequately pinpoint a location of a desired area to massage on an individual. The base 702 has a first side 730, a second side 732, a third side 734, and a fourth side 736. The first side 730 is opposite the second side 732, and the third side 734 is opposite the fourth side 736. The base 702 can be created with any desired flexible material including, but not limited to, polymers. The base 702 being created out of a flexible material allows the base 702 to flex in a manner that allows pressure to be applied to an individual while also forming to the shape of an individual's body. The entirety of the base 502 can be concave; however, in the illustrated embodiment, the base 502 has a relatively planar second surface 516 with a slight curve to make the base 502 concave.

The illustrated embodiment further has the elongate massage member 704. Although the illustrated embodiment shows three elongate massage members 704, the massager 700 can have any number of elongate massage members 704 such as, but not limited to, one, two, five, and nine. The elongate massage members 704 connect to the second surface 716 of the base 702. The elongate massage members 704 can be connected to the second surface 716 of the base 702 by any means such as, but not limited to, an adhesive or be integrally formed. The elongate massage members 704 can extend in a direction away from the second surface 716 of the base 702.

In the illustrated embodiment, the elongate massage members 704 are integrally formed with the base 702 and extend from the direction of the third side 734 of the base 702 to the direction of the fourth side 736 of the base 702. The elongate massage members 704 alternatively could extend along the length of the base 702 from the first end 720 to the second end 722. Also, the elongate massage members 704 could extend from the first end 720 to the second end 722. The elongate massage members 704 can be made from any desired material. However, it can be seen as beneficial to have the elongate massage member 704 made from a metallic material because metallic materials have a relatively low coefficient of friction when applied to the skin of an individual. Thus, this allows the massager 700 to glide across the skin of an individual. Further, the elongate massage members 704 can be tubular shaped; however, the elongate massage members 704 can have any desired shape so long as it allows the massager 700 to glide across an individual's skin. Also, the elongate massage members 704 could be liquid filled pads. For example, the liquid filled pads can be gel-filled pads. Further, the massager 700 has a first arch 705 and a second arch 707. The first arch 705 is on the second surface 716 of the base 702 adjacent the second side 732 of the base 702. The second arch 707 is on the second surface 716 of the base 702 adjacent the first side 730 of the base 702. The first arch 705 and the second arch 707 extend in a direction away from the second surface 716.

In the illustrated embodiment, the motor 706 is disposed in the base 702 at a location where the extension 708 comes in contact with the base 702. The motor 706 can be disposed in or on any desired portion of the base 702 including, but not limited to, a central portion 742 of the base 702, the extension 708, or the grip 710. The motor 706 is adapted to produce vibration that is transferred through the base 702 to the elongate massage member 704. The motor 706 can have any desired number of speeds. However, the illustrated embodiment has three speeds that are approximately 2,000 rpm, 3,000 rpm, and 6,000 rpm. The speeds could alternatively include, but are not limited to, approximately 500 rpm, 1,000 rpm, 3,000 rpm, 5,000 rpm, 7,000 rpm, and 9,000 rpm. Further, the massager 700 could have any desired number of motors 706 including, but not limited to, two, three, or four.

The illustrated embodiment further includes the extension 708. The extension 708 has an extension first end 744 and an extension second end 746. The extension first end 744 extends from the first surface 714 of the base 702 in a direction opposite the second surface 716 of the base 702. Further, the grip 710 is positioned at the extension second end 746. In the illustrated embodiment, the grip 710 is offset in relation to the extension 708 to allow a larger grip 710 for an individual to be able to easily hold the massager 700. However, the grip 710 can be any desired type of grip 710 that assists an individual in holding the massager 700. In alternative embodiments, the extension 708 can be eliminated, and the grip 710 can be attached or be integrally formed to the first surface 714 of the base 702. Further, the switch 712 is disposed in a grip top surface 750 of the grip 710. The switch 712 can be positioned on any desired side of the grip 710. The switch 712 includes a first button 756 and a second button 758. The first button 756 is configured to turn the motor 706 on and to cycle through a plurality of speed of vibrations when the first button 756 is pressed consecutively. The second button 758 is configured to turn the motor 706 off by pressing the second button 758. The switch 712 does not have to be on the grip 710; however, it is advantageous for the switch 712 to be on the grip 710 because it allows an individual to easily toggle the switch 712 between the on and off positions.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular examples disclosed herein have been selected by the inventors simply to describe and illustrate examples of the invention and are not intended to limit the scope of the invention or its protection, which is to be given the full breadth of the appended claims and any and all equivalents thereof.

We claim:

1. A massager, comprising:
a base, the base having a first surface, a second surface, a first side and a second side, the first surface opposite the second surface, the first side opposite the second side;
an elongate massage member, the elongate massage member connecting to the second surface of the base, the elongate massage member extending in a direction away from the second surface and extending from the first side of the base to the second side of the base,
a motor, the motor disposed in the base, the motor adapted to produce vibration that is transferred through the base to the elongate massage member; and
an extension, the extension having a first end and a second end, the first end of the extension extending from the first surface of the base in a direction opposite the second surface of the base;
a grip, the grip positioned at the second end of the extension; and
a switch, the switch disposed in the grip, the switch having an on and off position, the switch configured to turn the motor on when in the on position and to turn the motor off when in the off position.

2. The massager of claim 1, wherein the base has a length, the length extending from a first end of the base to the second end of the base, the length being approximately 3.5 inches to approximately 7 inches.

3. The massager of claim 2, wherein the base has a width, the width extending from a third end of the base to a fourth end of the base, the width being approximately 2.75 inches to approximately 6 inches.

4. The massager of claim 1, wherein the base has a length and a width, the length extending from a first end of the base to a second end of the base, the width extending from a third end of the base to a fourth end of the base, the length being approximately 3.5 inches and the width being approximately 2.75 inches.

5. The massager of claim 1, wherein the base has a length and a width, the length extending from a first end of the base to a second end of the base, the width extending from a third end of the base to a fourth end of the base, the length being approximately 5 inches and the width being approximately 4.5 inches.

6. The massager of claim 1, wherein the base has a length and a width, the length extending from a first end of the base to a second end of the base, the width extending from a third end of the base to a fourth end of the base, the length and the width being approximately the same.

7. The massager of claim 1, wherein the elongate massage members are partially disposed within the base.

8. The massager of claim 1, further comprises a plurality of elongate massage members.

9. The massager of claim 8, wherein the plurality of elongate massage members are partially disposed within the base.

10. The massager of claim 1, wherein the base is flexible.

11. The massager of claim 1, the base further having a concave wing, the concave wing defining the first side, the concave wing extending in a direction opposite the first surface; and
a second concave wing, the second concave wing defining the second side of the base.

12. The massager of claim 1, wherein the motor has three speeds.

13. The massager of claim 12, wherein the three speeds are approximately 2,000 rpm, 4,000 rpm, and 6,000 rpm.

14. The massager of claim 1, further comprising a second motor.

15. The massager of claim 14, wherein the motor has a different speed than the second motor.

16. The massager of claim 15, wherein the motor has a speed of approximately 2,000 rpm and the second motor has a speed of approximately 4,000 rpm.

17. The massager of claim 16, where the motor and the second motor can operate independently of each other and together.

18. The massager of claim 14, wherein the switch has a first button, a second button, a third button, and a fourth button, the first button configured to turn off the motor and the second motor, the second button configured to turn on the motor, the third button configured to turn on the second motor, the fourth button configured to turn on the motor and the second motor.

19. A massager, comprising:
A base, the base having a first surface, a second surface, a first side, and a second side, the first surface opposite the second surface, the first side opposite the second side, the base having a first concave wing and a second concave wing, the first concave wing defining the first side and the second concave wing defining the second side, the first concave wing and the second concave wing extending in a direction opposite the first surface;
a plurality of elongate massage members connecting to the second surface of the base, the plurality of elongate massage members extending in a direction away from the second surface and extending away from the first side of the base to the second side of the base;
a motor, the motor disposed in the base, the motor adapted to produce vibration that is transferred through the base to the plurality of elongate massage members;
an extension, the extension having a first end and a second end, the first end of the extension extending from the first surface of the base in a direction opposite the second surface of the base;
a grip, the grip positioned at the second end of the extension; and
a switch, the switch disposed in the grip, the switch having an on and off position, the switch configured to turn the motor on when in the on position and to turn the motor off when in the off position.

20. A method of performing self treatment for enhancing lymphatic drainage, comprising:
administering radiation to an affected area;
placing a massager at the affected area, the massager comprising:
a base, the base having a first surface, a second surface, a first side, and a second side, the first surface opposite the second surface, the first side opposite the second side, the base having a first concave wing and a second concave wing, the first concave win defining the first side and the second concave wing defining the second side, the first concave wing and the second concave wing extending in a direction opposite the first surface,
a plurality of elongate massage members, the plurality of elongate massage members connecting to the second surface of the base, the plurality of elongate massage members extending in a direction away from the second surface and extending from the first side of the base to the second side of the base;
a motor, the motor disposed in the base, the motor adapted to produce vibration that is transferred through the base to the plurality of elongate massage members;
an extension, the extension having a first end and a second end, the first end of the extension extending from the first surface of the base in a direction opposite the second surface of the base;
a grip, the grip positioned at the second end of the extension;
a switch, the switch disposed in the grip, the switch having an on and off position, the switch configured to turn the motor on when in the on position and to turn the motor off when in the off position;
choosing a speed of vibration;
turning the massager on;
applying pressure to the massager; and
massaging the affected area with the massager.
